(19) United States
(12) Patent Application Publication
(10) Pub. No.: US 2020/0123104 A1
(43) Pub. Date: Apr. 23, 2020

(54) PHARMACEUTICAL AGENT THAT BINDS THE P2X7 RECEPTOR

(71) Applicant: Indiana University Research and Technology Corporation, Indianapolis, IN (US)

(72) Inventors: Hamideh Zarrinmayeh, Carmel, IN (US); Mark Alan Green, Carmel, IN (US)

(73) Assignee: Indiana University Research and Technology Corporation, Indianapolis, IN (US)

(21) Appl. No.: 16/626,723

(22) PCT Filed: Jun. 28, 2018
(86) PCT No.: PCT/US2018/039923

§ 371 Date: Dec. 26, 2019

Related U.S. Application Data
(60) Provisional application No. 62/526,210, filed on Jun. 28, 2017.

(51) Int. Cl. C07D 207/28; G01N 33/534; A61K 51/04; G01N 33/60

(52) U.S. Cl. CPC C07D 207/28; G01N 33/60; A61K 51/0446; G01N 33/534

(57) ABSTRACT

Disclosed are P2X7 receptor ligands having the formula (I):

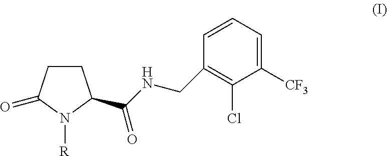

in which R=—(CH₂)ₙ—(O—CH₂CH₂)ₘ—Oₗ—(CH₂)ₚ—X, l=0-1, m=0-6, n=2-3, p=0-2, and X=Br, Cl, F or ¹⁸F. In illustrative embodiments, R=—(CH₂)ₙ, and n=2-3. The receptor ligands are used in methods related to conditions related to the P2X7 receptor. The non-radioligands are useful for modulating inflammation conditions associated with the P2X7 receptor. The radioligands, containing ¹⁸F, are useful for as radiotracers in imaging processes, and thus for the detection and evaluation of inflammation and therapies. Also provided are processes for preparation of these P2X7 receptor ligands.

### BACKGROUND

Chronic inflammation is characterized by sustained activation of microglia and overexpression of the purinergic receptor P2X7 on activated microglia.

Early evaluation and detection of inflammation is critical for potential prevention of further damage and monitoring possible therapy in some cases. Positron emission tomography (PET) offers potentially unparalleled sensitivity for non-invasive imaging to detect biomarkers of regional tissue inflammation. Thus far, the most used PET radiopharmaceuticals are ligands that bind to the Translocator Protein (TSPO), formerly called the Peripheral Benzodiazepine Receptor (PBR), associated with the mitochondria of activated macrophages and microglia.

The radioligands, [¹¹C]PBR28 and [¹¹C](R)-PK11195, have been used as tools in both animal models and human subjects in an attempt to assess inflammation through TSPO binding. Currently, the most widely-used, PET radiopharmaceutical for imaging evaluation of inflammation is [¹¹C]PBR28. Investigators are actively employing [¹¹C]PBR28 as a tool for assessment of inflammation in disorders associated with chronic inflammation including neuroinflammation conditions such as Alzheimer's disease and traumatic brain injury, as well as for detection of the inflammatory component of neurofibroma.

Unfortunately, in humans, [¹¹C]PBR28 has been found to exhibit high inter-subject variability in binding affinity, with a genetic polymorphism of the TSPO target resulting in population stratification into high-affinity binders, mixed-affinity binders, and low-affinity binders. Thus, the characteristics of TSPO as a molecular target appear suboptimal for the ultimate objective of quantifying regional inflammation.

The P2X7 receptor, a member of a P2X-family of purinergic receptors, is expressed on cells of hematopoietic origin. Within the central nervous system, functional P2X7 receptors are found on microglia, Schwann cells, and astrocytes.

Additionally, P2X7 receptors are rapidly upregulated by inflammatory stimuli to these immune cells, making the P2X7 receptor a potentially useful target in the design and development of PET agents intended for non-invasive evaluation of inflammation.

An increasing number of studies suggest that over-expression of the P2X7 receptor is strongly associated with inflammation. Thus, the P2X7 receptor represents an attractive inflammation-associated molecular target for PET imaging, due to its selective association with activated cells of the immune system. Additionally, the P2X7 receptor can be targeted with high-affinity low-molecular-weight ligands that are able to penetrate the blood-brain barrier, as required if a suitable PET radiopharmaceutical is to reach the immune cells of the central nervous system.

[¹¹C]GSK1482160 is a negative allosteric modulator of P2X7 having the structure:

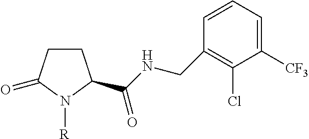

in which R=—¹¹CH₃. It readily crosses the blood-brain barrier, and has a P2X7 receptor affinity (KB) of 32 ng/mL (95 nM). Using high-specific-activity ¹¹C-GSK1482160 and human embryonic kidney cell lines expressing human P2X7R (HEK293-hP2X7R), we have measured receptor density of 3.0±0.1 pmol/mg and K=1.15±0.12 nM, with an association constant kon=0.231±0.015 min⁻¹·1 nM⁻¹, dissociation constant koff=0.255±0.016 min⁻¹, and binding potential of 1.03±0.21. While [¹¹C]GSK1482160 is a promising agent for research assessment of P2X7 receptor expression, the 20-minute half-life of ¹¹C is intrinsically incompatible with routine distribution to allow widespread use.

Improved imaging tools for reliable and non-invasive assessment of inflammation are needed for examining the role of inflammatory processes in diseases and disorders associated with inflammation and for assessment of responses to experimental therapies. Therefore, there is an urgent need to identify a more efficient and specific PET ligand to detect inflammation.

### SUMMARY

According to a first broad aspect, the presently disclosed subject matter provides compounds having the formula (I):

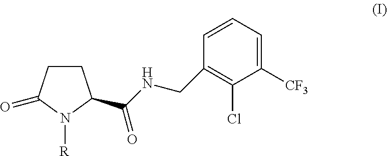

in which:

R=—(CH₂)ₙ—(O—CH₂CH₂)ₘ—Oₗ—(CH₂)ₚ—X,

l=0-1,

m=0-6,

n=2-3,

p=0-2, and

X=Br, Cl, F or ¹⁸F,

or a pharmaceutically-acceptable salt or solvate thereof. Illustrative embodiments include those in which n=2 or 3. In further embodiments, R=—(CH₂)ₙ—X, n=2-3, and X=Br, Cl, F or ¹⁸F. These compounds are used in various methods, including in combination with pharmaceutically-acceptable carriers.

According to another broad aspect, there are provided pharmaceutical compositions comprising a compound of formula (I), or a pharmaceutically-acceptable salt or solvate thereof, and a pharmaceutically-acceptable carrier. In exemplary embodiments, R=—(CH₂)ₙ—X and n=2-3. The compositions are useful in a variety of methods.

In another aspect, the compositions comprise compounds in which X is ¹⁸F and the radiopharmaceutical compositions are used in methods for performing positron emission tomography (PET), for imaging tissue inflammation, and/or for evaluating the effectiveness of a therapy.

The disclosed subject matter further provides methods and systems for delivering the ligand compounds and compositions into the body of a subject. For example, in one embodiment the system comprises the binding ligand within an injection fluid contained in an injectable delivery device. The injection fluid comprises the pharmaceutical composition, which may further comprise a pharmaceutically-acceptable carrier. The binding ligands and ligand compositions are in a form suitable for animal administration.

### BRIEF DESCRIPTION OF THE DRAWINGS

The accompanying drawings, which are incorporated herein and constitute part of this specification, illustrate exemplary embodiments of the disclosed subject matter, and together with the general description given above and the detailed description given below, serve to explain the features of the disclosed subject matter.

FIG. 1 provides a Scheme 1 illustrating the synthesis of illustrative ligands for binding with the P2X7 receptor as disclosed herein.

FIG. 2 provides a Scheme 2 illustrating the synthesis of illustrative ligands for binding with the P2X7 receptor, and particularly compounds designated as IUR-1601 and IUR-1602, as well as the related ethyl chloride and ethyl bromide.

FIG. 3 provides a Scheme 3 showing exemplary synthesis of representative P2X7 radioligands [¹⁸F]IUR-1601 and [¹⁸F]IUR-1602.

FIG. 4 is a graph illustrating the binding of IUR-1601 in an in vitro assay according to an exemplary embodiment of the presently disclosed subject matter.

FIG. 5 is an image showing a disclosed ligand composition formulated in the dosage form of a softgel according to an exemplary embodiment of the present invention.

FIG. 6 is an image showing a disclosed ligand formulated in the dosage form of a hard capsule according to an exemplary embodiment of the present invention.

FIG. 7 is an image showing a disclosed ligand formulated in the dosage form of a tablet according to an exemplary embodiment of the present invention.

FIG. 8 is an image showing a disclosed ligand formulated in the dosage form of chewable tablet according to an exemplary embodiment of the present invention.

FIG. 9 is an image showing a disclosed ligand formulated in the dosage form of caplet according to an exemplary embodiment of the present invention.

FIG. 10 illustrates a delivery apparatus for delivering a disclosed ligand according to an exemplary embodiment of the presently disclosed subject matter suitable for parenteral administration.

### DESCRIPTION

### Definitions

Where the definition of terms departs from the commonly used meaning of the term, applicant intends to utilize the definitions provided below, unless specifically indicated.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise.

For purposes of the present invention, it should be noted that to provide a more concise description, some of the quantitative expressions given herein are not qualified with the term “about” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present invention, the term “comprising”, the term “having”, the term “including,” and variations of these words are intended to be open-ended and mean that there may be additional elements other than the listed elements.

For purposes of the present invention, the term “analog” refer to one of a group of chemical compounds that share structural and/or functional similarities but are different in respect to elemental composition. A structural analog is a compound having a structure similar to that of another one, but differing from it in respect of one or more components, such as one or more atoms, functional groups, or substructures, etc. Functional analogs are compounds that have similar physical, chemical, biochemical, or pharmacological properties. Functional analogs are not necessarily also structural analogs with a similar chemical structure.

For purposes of the present invention, the term “ligand” refers to a compound that binds to a receptor. Even though these compounds are acting as antagonists; the ligand could be either agonist or antagonist.

For purposes of the present invention, the term “in vivo imaging” refers to those techniques that non-invasively produce images of all or part of an internal aspect of an animal subject, e.g., a mammalian subject, including humans.

For purposes of the present invention, the term “pharmaceutically acceptable” refers to a compound or drug approved or approvable by a regulatory agency of a federal or a state government, listed or which may be listed in the U.S. Pharmacopeia or in other generally recognized pharmacopeia for use in animals, e.g., mammals, including humans. A “pharmaceutically acceptable carrier” is a carrier which is physiologically acceptable to the subject while retaining the therapeutic properties of the pharmaceutical composition with which it is administered. One exemplary pharmaceutically acceptable carrier is physiological saline. Other physiologically acceptable diluents, excipients, carriers, or adjuvants and their formulations are known to one skilled in the art.

For purposes of the presently disclosed subject matter, the term “radioligand” refers to a radiolabeled ligand that is used, inter alia, for imaging, diagnosis, or evaluation, or as a tool for research-oriented study of the receptor systems of a body. In a neuroimaging application the radioligand is injected into the pertinent tissue, or infused into the bloodstream. It binds to its receptor. When the radioactive isotope in the ligand decays it can be measured by positron emission tomography (PET) or single photon emission computed tomography (SPECT). In in vivo systems it is often used to quantify the binding of a test molecule to the binding site of the receptor. The receptor-avid radioligand will be selectively retained in tissue regions expressing the receptor, providing image contrast between receptor-expressing and non-expressing sites, allowing diagnostic detection as well as possible quantification of available levels of receptor.

For purposes of the presently disclosed subject matter, the term “radiotracer” and the term “radioactive tracer” refer to a chemical compound in which one or more atoms have been replaced by a radioisotope to allow easier detection and measurement. The radioisotope is usually a short-lived positron emitting radioisotope. A radioactive tracer can be used to track the distribution of a substance within a natural system such as a cell or tissue, or as a flow tracer to track fluid flow. Radioactive tracers form the basis of a variety of imaging systems, such as PET scans and SPECT scans, and can be used in nuclear medicine imaging.

The term “subject” or “patient” as used herein, refers to an animal which is the object of treatment, observation or experiment. By way of example only, a subject may be, but is not limited to, a mammal including, but not limited to, a human.

### Compounds

There is a clear need for a PET radiopharmaceutical that can robustly quantify levels of inflammation. The P2X7 receptor is an attractive molecular target for detection of activated immune cells. The agents disclosed herein are novel compounds suitable for targeting the P2X7 receptor in vivo.

GSK1482160 is an allosteric P2X7 receptor modulator known to have high P2X7 receptor affinity and blood-brain barrier permeability. We previously developed [¹¹C]GSK1482160 as a radiopharmaceutical for targeting the P2X7 receptor. The compounds described herein are a series of compounds designed to retain the P2X7 receptor affinity of GSK1482160, but with structural modification to facilitate alternative radiolabeling strategies; control receptor affinity; and optimize biodistribution and pharmacokinetics for imaging applications.

The compounds disclosed herein include non-radioligands and ¹⁸F radioligands. The ligands without radio-labeling are useful, inter alia, in methods of treatment of conditions involving the P2X7 receptor. The ligands labeled with ¹⁸F are useful, inter alia, for kinetic imaging, as in PET, as well as in therapy of conditions involving the P2X7 receptor and evaluation of such therapy.

Disclosed herein are compounds having the following formula (1):

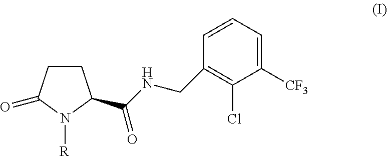

in which:

R=—(CH₂)ₙ—(O—CH₂CH₂)ₘ—Oₗ—(CH₂)ₚ—X,

n=2-3,

m=0-6,

l=0-1,

p=0-2, and

X=Br, Cl, F or ¹⁸F,

or a pharmaceutically acceptable salt or solvate thereof.

In illustrative embodiments, the compounds include those in which X=F or ¹⁸F, and alternatively in such embodiments in which n=2. In alternative embodiments, l, m and p are each zero, such that R=—(CH₂)ₙ—X, n=2-3 and X=Br, Cl, F or ¹⁸F. By way of example, these compounds include the following compounds in which n=2 and 3, respectively:

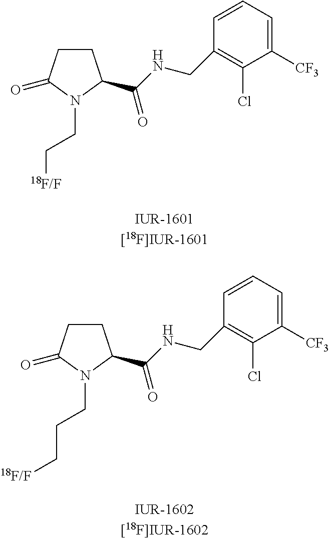

As indicated, the compounds where n=2 or 3 and X=F provide the compounds designated as IUR-1601 and IUR-1602, respectively. In the same manner, the compounds where n=2 and 3, and X=¹⁸F, yield the compounds [¹⁸F] IUR-1601, and [¹⁸F] IUR-1602, respectively. The Br and Cl analogs are also disclosed.

### Preparation of Non-Radioligands

In an illustrative embodiment, R=(CH₂)ₙ—X, and the P2X7 receptor ligands have the following formula (II):

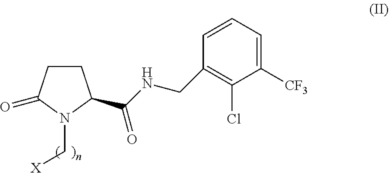

in which n=2-3 and X=Br, Cl, F or ¹⁸F.

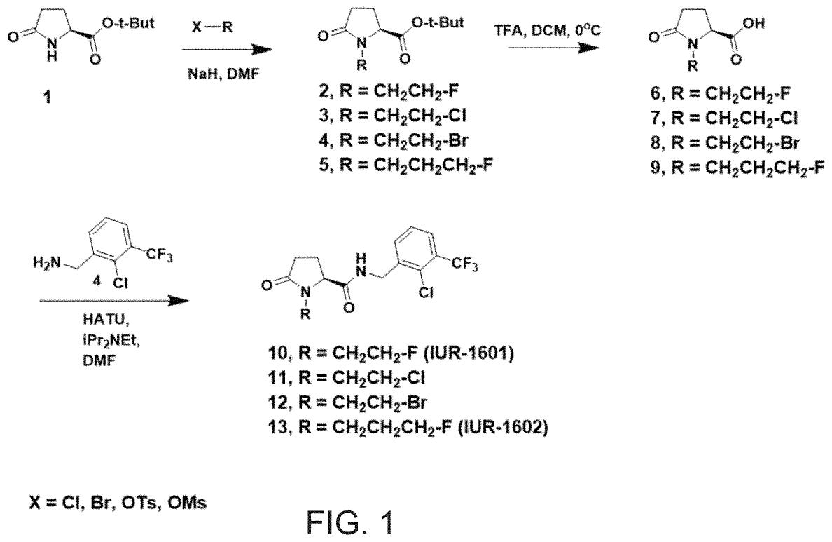

Shown in FIG. 1 is an exemplary Scheme 1 for the preparation of compounds having the formula (II). As shown in Scheme 1, commercially available starting material tert-butyl (S)-5-oxopyrrolidine-2-carboxylate (1) was dissolved in N,N-dimethylformamide (DMF) and the solution was treated with sodium hydride (NaH). The reaction mixture was stirred at room temperature (RT) for 30 minutes and treated with fluoroethylbromide (n=2). Alternatively, the starting material is alkylated with tosylate, mesylate, nosylate, bromo, iodo, and/or chloro precursors.

The resulting mixture was stirred at RT for 2 h. The reaction was quenched with water (1×). Organic product was extracted with ethyl acetate (3×), dried and concentrated under vacuum to yield the desired crude products which were purified with column chromatography to yield the corresponding N-fluoroethyl tert-butyl (S)-(5-oxopyrroline-2-carbonyl)carbamate ester (2). Intermediates 3-4 were prepared by alkylation of starting material 1 with 1-bromo, 2-chloroethane and 1, 2-dibromoethane respectively. Intermediate 5 was prepared by alkylation of starting material 1 with 1-bromo, 3-fluoropropane. Ester intermediates (2-5) were hydrolyzed with trifluoroacetic acid (TFA) in dichloromethane (DCM) to give the corresponding acids 6-9. Without further purification, acids 6-9 were coupled with 2-chloro-3-(trifluoromethyl)benzylamine (TFBA) using coupling reagents 1-ethyl-3-(3-dimethylaminopropyl)-carbodiimide hydrochloride (EDCA) or 2-ethoxy-1-ethoxycarbonyl-1,2-dihydroquinoline (EEDQ) to yield the amides 10-13:
((S)—N-(2-chloro-3-(trifluoromethyl)benzyl)-1-(2-fluoroethyl)-5-oxopyrrolidine-2-carboxamide) (10),
((S)—N-(2-chloro-3-(trifluoromethyl)benzyl)-1-(2-chloroethyl)-5-oxopyrrolidine-2-carboxamide) (11),
((S)—N-(2-chloro-3-(trifluoromethyl)benzyl)-1-(2-bromoethyl)-5-oxopyrrolidine-2-carboxamide) (12), and
((S)—N-(2-chloro-3-(trifluoromethyl)benzyl)-1-(2-fluoropropyl)-5-oxopyrrolidine-2-carboxamide) (13)
   The products were obtained in reasonable yield after purification.

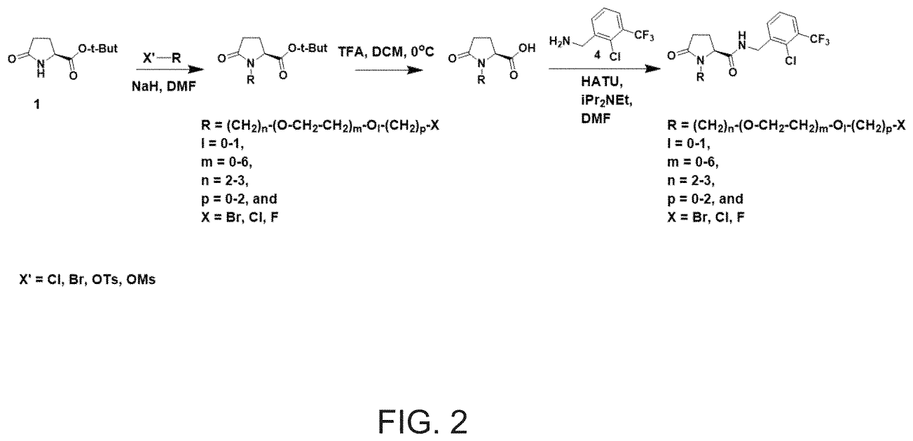

Shown in FIG. 2 is an exemplary Scheme 2 for the preparation of analog compounds having the formula (1). This Scheme 2 parallels Scheme 1 but with substitution of appropriate other reactants as will be readily appreciated by those of ordinary skill in the art.

### Preparation of Radioligands

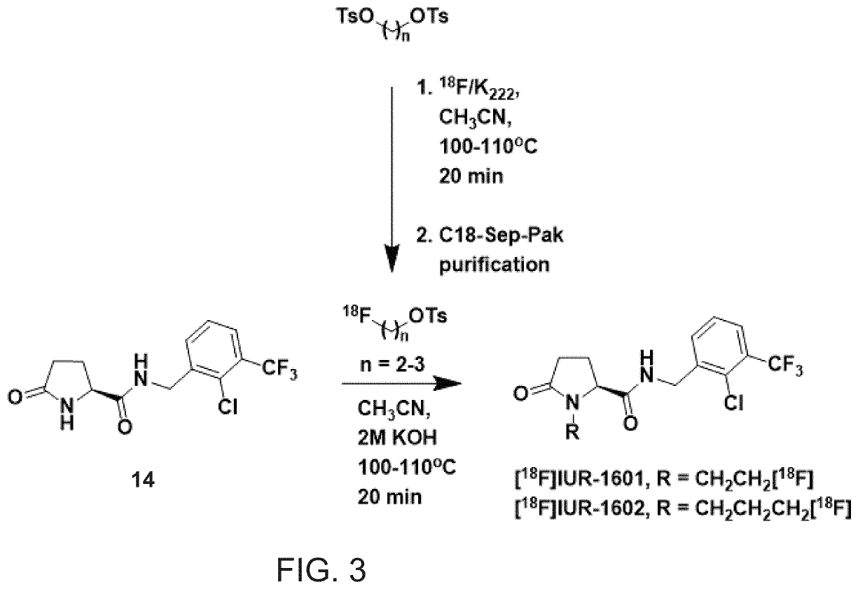

Referring to FIG. 3, there is shown an exemplary Scheme 3 for the synthesis of the disclosed radioligands, using [¹⁸F]IUR-1601 and [¹¹F]IUR-1602 as examples. As shown in Scheme 3, 1,2-ditosylethane (n=2) and 1,3-ditosylpropane (n=3) were dissolved in acetonitrile (CH₃CN) and reacted with K[¹⁸F]/Kryptofix2.2.2 (¹⁸F/K₂₂₂) at 100-110° C. for 20 minutes to form the radiolabeled precursor 2-[¹⁸F], 1-tosylethane [and 3-[¹⁸F], 1-tosylpropane] which were subsequently purified by a C18 Plus Sep-Pak and added to a mixture solution of intermediate and 2M potassium hydroxide in acetonitrile. The intermediate (compound 14 in FIG. 3) may be prepared, for example, as described at (https://www.sciencedirect.com/science/article/pii/S0960894X15002176). The reaction mixture was stirred at 100-110° C. for 20 minutes to give the target tracers [¹⁸F]IUR-1601(n=2) and [¹⁸F]IUR-1602 (n=3), which were purified by semi-preparative RP HPLC (C18 column) combined with solid phase extraction (SPE) using a C18 Plus Sep-Pak cartridge.

The overall radiochemical yield for the two-step radiosynthesis of [¹⁸F]IUR-1601 was 1-10% decay corrected to end bombardment (EOB) based on the H[¹⁸F]F. The chemical and the radiochemical purity were >95% and >99%, respectively, and were determined by radio-HPLC through γ-ray (PIN diode) flow detector.

The analytical RP HPLC system used to monitor all organic synthetic and radiosynthetic reactions included a Prodigy (Phenomenex) 5 μm C-18 column, 4.6×250 mm, a gradient mobile phase (40-80%) CH3CN/3 mM HCOONH4, flow rate 1.8 mL/min; UV (270 nm) and γ-ray (PIN diode) flow detectors.

The radiosynthesis was performed in a self-designed automated multi-purpose [¹⁸F]-radiosynthesis module. This radiosynthesis module facilitates the overall design of the reaction, purification and reformulation capabilities in a fashion suitable for adaption to preparation, purification, and formulation for human doses while concurrently limiting operator exposure to ionizing radiation.

To study a mechanism in humans using an agent suitable for commercial distribution, there is a need for a radioligand with a longer half-life than carbon 11. The disclosed novel [¹⁸F]IUR-1601 and [¹⁸F]IUR-1602 are radioligands that serve this goal, since they offer the longer half-life of −[¹⁸F] ti/2=109.8 min. These compounds are therefore suitable for commercialization through established nation-wide infrastructure for production and distribution of ¹⁸F-radiopharmaceutical drug products. Using ¹⁸F as the radiolabel provides a radiopharmaceutical that can be regionally produced and made available to hospitals throughout the U.S. following the model of the existing commercial manufacturing infrastructure for widely-used ¹⁸F-fluorodeoxyglucose (FDG). These characteristics make [¹⁸F]IUR-1601 and [¹⁸F]IUR-1602 valuable as radiotracers of P2X7 receptor density used as a biomarker of inflammation.

### Characterization of Products

The non-radioactive products are fully characterized by using standard analytical methods—¹H and ¹³C NMR spectroscopy, UV/Vis spectroscopy, and mass spectrometry, in addition to establishing their behavior under the HPLC conditions anticipated for use in evaluation of radiopharmaceutical chemical and radiochemical purity and specific activity.

Physicochemical characterization of the purified products [¹⁸F]IUR-1601 and [¹⁸F]IUR-1602 involves (direct measurement of the radiopharmacetical's octanol/water partition coefficient to measure lipophilicity. Binding to serum proteins is independently measured by ultrafiltration. These properties are helpful for quantitative prediction of the radiopharmaceutical's ability to passively diffuse across the blood brain barrier (ideally, 0.5<log P<3.0), and prediction of differences in bioavailability that may occur between species.

### Ligand Methods

The disclosed P2X7 receptor ligands are useful in a variety of methods based on their affinity, biodistribution, pharmacokinetic and other properties. The methods may include in vitro and in vivo evaluation of inflammation.

In one aspect the P2X7 ligand compounds, which may be combined with a pharmaceutically acceptable carrier, are administered to a subject either orally or by other routes, to treat a medical condition, e.g, inflammation, associated with the P2X7 receptor. The ligand compound for this purpose may be any defined by the previously identified formula (1). The ligand compound need not be radio-labeled for this use. In particular embodiments the ligand compound has the formula (I) in which R=—(CH₂)ₙX. Preferably, the ligand compound has the formula (I) where R=—(CH₂)ₙX, n=2-3, and X=Br, Cl or F. In a particular embodiment n=2 or 3 and X=F. Once administered, the ligands travel to the site of the P2X7 receptor and bind with the P2X7 receptor. This, for example, acts to modulate the inflammation.

In another aspect a method is provided for imaging tissue using the radioligands disclosed herein as a radiotracer that binds to the P2X7 receptor. The ligand compounds are described by formula (I) in which X=¹⁸F. In particular embodiments, n=2 or 3. The ligand compound is administered in a manner to contact the tissue in which the P2X7 receptors are located to cause at least a portion of the P2X7 receptors contained in the tissue. The tissue is then imaged in known fashion. In a particular embodiment the tissue is imaged using PET.

The imaging may be used to detect and/or define regional tissue expression of P2X7 receptors (associated with a number of diseases); detect, assess and/or evaluate tissue inflammation; and/or assess the effectiveness of a treatment. Diagnostic clinical utility for these agents can take the form of reliable detection of regional inflammation, or reliable monitoring of response to therapies that diminish regional inflammation or that require demonstration of P2X7 receptor occupancy.

### Verification of Products

The radiosynthetic methods for an agent judged suitable for translation to humans must allow delivery of product meeting the requirements of USP<823>. Post-production, pre-release quality control steps include GC analysis for residual reaction solvents, plus HPLC analysis with UV and radiation detection to confirm the radiochemical identity of the product and to quantify product radiochemical purity, specific activity, and chemical purity. Pre-release product testing also includes measurement of the final solution pH, verification of radionudclide identity with a rapid half-life measurement testing for bacterial endotoxin, and bubble point testing to verify the integrity of the sterilizing filter. Doses undergo a USP sterility test which by its duration must be performed as a retrospective (post-release) quality assurance test.

For compounds delivering promising results in the biological assays, radiosynthetic methods are refined to optimize reaction time, temperature, and semi-preparative HPLC conditions (flow rate and solvent composition), tracer prepared in optimized synthetic conditions is used in companion experiments to optimize and fully validate the required radioanalytical HPLC conditions for assessment of radiochemical purity and product specific activity. Once optimized synthesis and QC conditions are established, final SOPs for both tracer synthesis and quality control testing are prepared, a final Batch Record document is generated, and methods for the process validation studies required in support of advancement to human studies are implemented.

Measurement of Receptor Affinity and Estimation of Binding Potential.

Receptor density (Bmax) is determined by a saturation approach as employed with each P2X7-receptor ligand employing [¹¹C]GSK1482160 as a convenient reference ligand that is expected to bind in the same region of the receptor. Using the cold fluoroalkyl standards (IUR-1601 and IUR-1602), binding is measured using cell membrane homogenate from human embryonic kidney cells (HEK) transfected with human P2X7 gene (B'SYS GmbH, Switzerland). Samples and control wells are run in triplicate at each concentration. After equilibrium has been reached, separation of bound from free [¹¹C]GSK1482160 radioligand is performed using a 96-well filtration apparatus. To estimate receptor association (kₒₙ) and dissociation (k_{off}) rates, an association/dissociation kinetic experiment is performed. Association is measured as a function of time using cell membrane homogenate from HEK-P2X7 cells incubated with 2-4 concentrations of the ¹⁸F-radioligand. Dissociation is measured in the presence of excess authentic standard once at steady state according to published methods. Radioactivity for both studies is measured on the filters (or filter plates) using a Perkin-Elmer TopCount Data is analyzed and plotted using GraphPad Prism to obtain Bmax and Kd values for the compound.

Determination of Bp (kon/koff) is performed using an association/dissociation kinetic model. In order to evaluate brain binding, ex vivo autoradiography is performed in mice exposed to saline or LPS (5 mg/kg) IP injections. Intraperitoneal LPS administration has previously been shown to activate microglia via IL-6, TNF-α and IL10 mediated response, and therefore provides a predictable and dose dependent mechanism to generate ex vivo samples for evaluation of P2X7 binding Brain sections are incubated in 2 concentrations of radioligand, washed 6 times, and the sections are then used to expose a phosphor plate along with standards, and scanned on a GE Typhoon FLA7000 IP (GE Medical Systems, USA). Secondary confirmation is performed via Immunofluorescence Assay (IFA) using a CD40 antibody conjugated to NIR-fluorophore and scanned on a LICOR Odyssey (LICOR, USA). Image analysis and quantification are performed using MCID Analysis software (MCID, UK).

In Vivo Measurement of the Biodistribution and Pharmacokinetics of the Radiolabeled P2X7 Receptor Ligands in Animal Models.

Animal studies assess each radiopharmaceutical's whole body distribution and clearance kinetics, and confirm P2X7-receptor-specific targeting in vivo. The animal studies also establish the correlation between radiopharmaceutical targeting and levels of regional P2X7 receptor expression established by post-mortem tissue assays; examine the correlation between radiopharmaceutical uptake and post-mortem assays of reference markers of inflammation; and assess selectivity for distinguishing inflamed and normal tissue.

Validation of ¹⁸F-P2X7 Ligand Characteristics and Performance In Vivo.

The in vitro assays described above verify affinity and selectivity for radiopharmaceutical binding to the P2X7 receptor. Agents are evaluated to confirm and characterize receptor-based targeting in vivo. The characterization of radiopharmaceutical performance in mice has four discrete objectives:

1) Screening biodistribution: Relevant to neuroinflammation-related applications, the ability of the radiopharmaceutical to penetrate the blood-brain barrier following intravenous administration is determined. More generally for inflammation elsewhere, assessment is made of uptake in major organs throughout the body as a function at a given time post administration. These data, combined with the in vitro binding and kinetics, are used to stratify the candidate ligands.

2) Biodistribution and pharmacokinetics: Determination of the radiopharmaceutical's tissue distribution in an exogenous model of inflammation with time. These data provide key information about the fate and distribution of the radiopharmaceutical during normal (i.e, saline) and inflammation (i.e. lipopolysaccharide) physiological states in mice. In addition, because these data are collected with time, estimates of tissue dosimetry and organ pharmacokinetics can be determined.

3) Target engagement and selectivity: Confirmation that the radiopharmaceutical effectively targets the P2X7 receptor is best accomplished by comparing the biodistribution observed in the presence, and absence, of competing high affinity ligands for the receptor. One uses a model that reliably expresses the receptor target so one can see selective uptake that is blocked when the radiotracer is co-administered with an excess of the unlabeled (competing) ligand.

Confirmation in vivo may use an animal model (or models) of inflammation including assessment of radiopharmaceutical kinetics, and the level of contrast that can be achieved between target (i.e., receptor-expressing) and non-target tissues through time. These data provide evidence of P2X7R engagement through time, and in the presence of excess unlabeled parent enable determination of selectivity of the ligand to the target

4) Target modulation (for therapeutic uses): Confirmation that the radiopharmaceutical effectively targets the P2X7R by reducing activated microglial density through colony-stimulating factor 1 receptor (CSF1R) inhibition. These data provide key evidence that the ligand binds to the P2X7R, and observed changes in PET signal are due to changes in microglial P2X7R expression in vivo.

Screening Biodistribution of ¹⁸F-Radiopharmaceutical in Mice.

To evaluate the organ exposure and the extent to which the candidate radiopharmaceuticals are able to cross the blood brain barrier (for evaluation of neuroinflammation) at steady state, quantitative data are used to define the overall distribution and brain penetration of the compound following intravenous (IV) administration at tracer doses. This can be performed either as an imaging study, in which PET images are acquired on individual animals at multiple time points (e.g., 1-2, 2-4, 4-8, 8-12, 12-20, 20-50, 50-80, 80-120, and 120-180 minutes' post-injection) after administration of ˜50-500 μCi of radiopharmaceutical. Alternatively, organ uptake can be directly quantified by animal sacrifice and harvesting of relevant organs and tissues for direct counting in a well-type gamma scintillation counter to quantify contained levels of the F-18 radiolabel. If organs/tissues are to be counted after resection, a lower level of radiopharmaceutical can be administered relative to the quantity required for imaging—thus, the ¹⁸F-radiopharmaceutical (˜1-20 pCi) is administered to conscious mice via intravenous injection into the tail vein. The amount of radioactivity administered to each animal is decay corrected and the biodistribution of the radiopharmaceutical is calculated as both a percentage of the injected dose per organ (% ID/organ) and a percentage of the injected dose per gram of tissue wet mass (% ID/g). Just prior to sacrifice, animals are anesthetized via isoflurane anesthesia (3-5%/balance medical oxygen) and euthanized via rapid decapitation at 30 minutes' post-injection (n=5/gender/tracer).

Assessment of ¹⁸F-Radiopharmaceutical Biodistribution, Pharmacokinetics, and Radiation Dosimetry in Mice.

For agent(s) where in vitro assays and screening biodistribution establish high affinity, selectivity, kinetics, and excellent in vivo tissue distribution, quantitative assessment of tissue distribution and pharmacokinetics of the selected radioligand(s) is performed. To enhance the expression of P2X7R, mice will be administered saline or 5 mg/kg lipopolysaccharide 72 hr prior to tracer administration as previously described. The tracer is administered in conscious mice via tail vein, and the mice are euthanized at 15, 30, and 120-minutes post-injection (n=5/gender/treatment/time). Relevant tissues and organs (blood, heart, lungs, liver, spleen, kidney, brain, muscle, brain, and bone) are excised, weighed, and counted to quantify the tissue uptake of the radiolabel. Although some of these tissues are not directly relevant to the goal of imaging inflammation-related P2X7-receptor binding of the radiopharmaceutical, they do provide critical information of the radiotracer fate needed to determine the organ dosimetry and primary clearance methods. Moreover, concurrent analysis of this expanded range of tissues provides the ability to estimate human organ and whole-body radiation dosimetry using the MIRD approach and OLINDA software package, in addition to accounting for the overall mass balance of the radiopharmaceutical(s) fate. To assess defluorination of the radiopharmaceutical, bone uptake (characteristic of ¹⁸F-fluoride) is used as a surrogate for assessing ligand degradation in vivo. Any compound showing extensive defluorination in vivo is rejected from further consideration, unless the nature of the metabolic pathway can be localized to a tissue such as the liver, and it can be shown that that metabolic pathway is specific to the rodent species but not apparent in the fate of the radiopharmaceutical with human cells in culture. For optimal performance as a tracer to image inflammation, the brain uptake of the radiopharmaceutical may be in the range of 0.1-10% of the injected dose at 2-5 minutes' post-injection, accompanied by rapid clearance of that radioactivity from the normal brain by 10-120-minutes post-injection. Brain uptake above 2% of the injected dose at 1-minute will indicate the tracer is very highly extracted in its first capillary transit and therefore at least initially exhibits perfusion-rate-limited brain uptake. While this does not necessarily limit the utility for mapping inflammation, perfusion-rate-limited brain penetration complicates modeling to quantify inflammation, because the tracer would initially simply map the pattern of cerebral perfusion, requiring tracer wash-out and delayed imaging to capture images reflecting the desired inflammation-mediated retention of radiotracer.

Radiopharmaceutical Assessment of Target Engagement and Receptor Selectivity in an Animal Model of Inflammation.

For characterization of the selected radiopharmaceutical's ability to bind to the P2X7-receptor in vivo, the 5×FAD mouse is a potential animal model that can be employed, since these animals exhibit age-dependent (brain) expression of P2X7 receptors. Data in the 5×FAD mouse, suggests that there is a 2-fold increase in P2X7R protein expression between 2 and 4 month and that this levels off by 6 month. Provided this, the ongoing breeding and phenotyping of the MODEL-AD consortium 5×FAD breeding pairs are used as an animal model of inflammation. The mice are bred, genotyped, and aged to the above time points. In order to assess the effects of age on P2X7R binding, groups of 5×FAD mice (n=10/gender/time) are imaged via simultaneous PET/MR imaging performed with a Bruker Biospec 9.4T/30 USR MRI outfitted with a Si 20/12 PET insert and a dedicated four-channel phased array mouse head coil (Bruker Biospin, Billerica, Mass.). To provide anatomical reference and assess grey matter density, a T2 weighted (T2W) 3D-SPACE MRI sequence is employed. For PET determination of P2X7R density, 150-250 uCi tracer is administered and calibrated listmode PET images are acquired concurrently with the MRI on the Biospec Si 20/12 PET insert for 60 min, and reconstructed into a dynamic image series using filtered-back-projection. In all cases, images are corrected for radionudclide decay, tissue attenuation, detector dead-time loss, and photon scatter according to the manufacturer's methods. Post-acquisition, all PET and MRI images are co-registered using a mutual information based normalized entropy algorithm with 6 degrees of freedom, and mapped to stereotactic mouse brain coordinates. To assess radiopharmaceutical specificity, a subset of mice (n=10/gender) at 6 months is administered excess unlabeled authentic standard IV and allowed to equilibrate for 5 min prior to radio tracer administration and PET/MRI imaging as described previously. Voxels of interest (V01) for 35 brain regions are extracted and kinetically modelled with a two compartment 5 parameter model and estimates of regional tissue perfusion, non-specific binding and specific binding and total volumes of distribution are computed. At the termination of the imaging study, brains are rapidly excised, sectioned hemi-coronal, slow frozen, embedded, and cyosectioned for ex vivo autoradiography.

Radiopharmaceutical Assessment of Target Modulation in an Animal Model of NeuroInflammation.

To confirm that the radiopharmaceutical effectively targets microglial P2X7R and that modulations can be determined by PET/MRI, 2 month old 5×FAD mice (10/gender/dose) are administered either normal chow (control) or chow treated with 1 or 2 doses of colony-stimulating factor 1 receptor (CSF1R) inhibitor (i.e. PLX5622 or PLX3397) for a minimum of 12 weeks. Mice are dynamically imaged via simultaneous PET/MRI, images reconstructed and coregistered, VOIs extracted, and regions kinetically modeled. Post mortem hemi-coronal brain sections are submitted for autoradiography, and P2X7R receptor density confirmed via WB. Western blot (WB) analysis are performed on the remaining hemi-coronal brain section to confirm the presence of P2X7R receptors.

Demonstration of utility for use in humans follows from knowledge that (1) the radiopharmaceutical has documented affinity for the receptor being targeted; (2) that the radiation dosimetry at the administered level of radioactivity will pose no safety problems; and (3) that the administered mass is going to be below the dose threshold where one could expect to see a pharmacological effect from the administered mass of agent. In total, translation to ¹⁸F-radiopharmaceutical imaging in humans is indicated by the ability of the ¹⁸F-N-fluoroalkyl compounds to effectively target the P2X7 receptor in vivo, and the P2X7-receptor-mediated localization correlates with independent measures of regional tissue inflammation.

Drug Properties. The high affinity for the P2X7 receptor demonstrated by the fluoroethyl compound, IUR-1601, makes clear that the related derivatives might also have significant affinity for the receptor. Preparation and characterization of those agents, and animal studies to examine structure-distribution relationships for those exhibiting high receptor affinity in vitro, represents a pathway for identifying an optimized agent that would merit translation to investigation in humans.

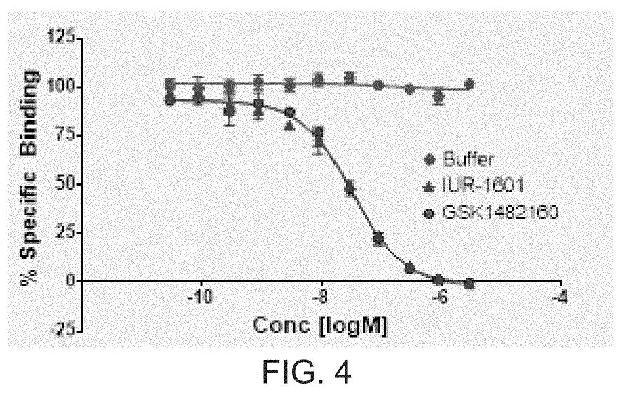

Applying described methods we have described previously, using cell membrane homogenate from human embryonic kidney cells (HEK) transfected with the human P2X7 gene and employing [¹¹C]GSK1482160, as a reference tracer, we have established that IUR-1601 has high affinity (Ki=4.31±0.92 nM and IC₅₀=7.17) for the P2X7 receptor, as needed for an ¹⁸F-labeled PET radiopharmaceutical. See, FIG. 4. The Ki for IUR-1601 is indistinguishable from the value of 5.14±0.85 nM that we have measured for [¹¹C]GSK1482160 in this same assay.

These results for the IUR-1601 N-fluoroethyl compound: (i) establish the synthetic accessibility of the class of novel N-fluoroalkyl compounds as P2X7-receptor ligands; and (ii) confirm the compounds to be good candidates for biological screening as likely high affinity ligands for the human P2X7 receptor.

In vivo measurement of the biodistribution and pharmacokinetics of the F-18 radiolabeled P2X7 receptor ligands in normal animals and models of inflammation indicate the radiopharmaceutical's whole body distribution, clearance kinetics, receptor affinity and non-specific binding to confirm the P2X7-receptor-specific targeting in vivo and establish the relationship between radiopharmaceutical specificity and regional P2X7 receptor expression.

### Compositions

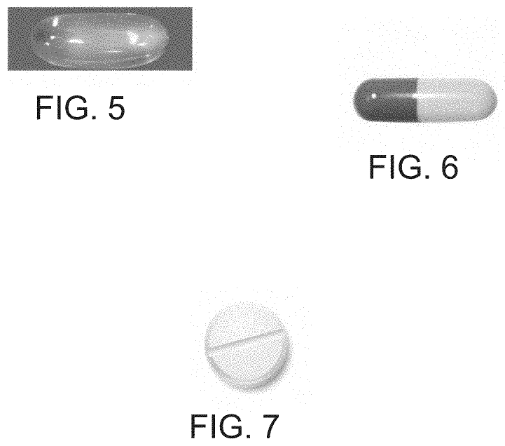

The radiopharmaceutical compositions disclosed herein comprise ligands which may be formulated with pharmaceutically acceptable carriers to provide a form suitable for oral or parenteral administration. For example, the P2X7 receptor ligands disclosed herein can be formulated in various dosage forms, including as a soft gel, hard capsule, tablet, chewable tablet or caplet, as shown in FIGS. 5-9, respectively. Alternatively, the ligand compounds and/or compositions can be administered by the subcutaneous, intramuscular, intravenous, transdermal, intranasal, rectal, ocular, topical, sublingual, buccal, or other routes.

The P2X7 receptor radioligands can also be delivered to a body of a subject via injection. The radiopharmaceutical composition can be formulated in a dosage form of an injection fluid and be loaded into an injectable device (e.g., a syringe) to inject into a subject's body. The radiopharmaceuticals of the present disclosure are formulated as sterile products suitable for parenteral administration, preferentially intravenous administration. A typical formulation is as a saline solution containing perhaps 5% ethanol as an excipient. FIG. 10 is an illustration of a treatment delivery apparatus (810) comprising an injectable drug delivery device (812) and a ligand composition disclosed herein in the dosage form of an injection fluid (814). The ligand compositions can be delivered via injection through the skin (820) of a subject and go into the body (822) of the subject. The injectable drug delivery device can stay outside (824) of the subject's body.

The many features and advantages of the invention are apparent from the detailed specification, and thus, it is intended by the appended claims to cover all such features and advantages of the presently disclosed subject matter which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

While the presently disclosed subject matter has been disclosed with reference to certain embodiments, numerous modifications, alterations, and changes to the described embodiments are possible without departing from the sphere and scope of the presently disclosed subject matter, as defined in the appended claims. Accordingly, it is intended that the presently disclosed subject matter not be limited to the described embodiments, but that it has the full scope defined by the language of the following claims, and equivalents thereof.

1. A compound having the formula (I): 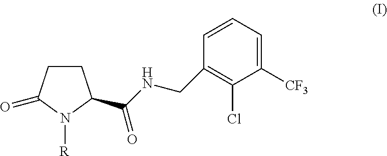
in which:
R=—(CH₂)ₙ—(O—CH₂CH₂)ₙ—Oₗ—(CH₂)ₚ—X,
l=0-1,
m=0-6,
n=2-3,
p=0-2, and
X=Br, Cl, F or ¹⁸F,
or a pharmaceutically acceptable salt or solvate thereof.

2. The compound of claim 1 in which X=F.

3. The compound of claim 2 in which n=2.

4. The compound of claim 1 in which X=¹⁸F.

5. The compound of claim 4 in which n=2.

6. The compound of claim 1 in which R=—(CH₂)ₙ—X.

7. The compound of claim 6 in which n=2.

8. The compound of claim 6 in which X=F.

9. The compound of claim 8 in which n=2.

10. The compound of claim 8 in which n=3.

11. The compound of claim 1 in which R=—(CH₂)ₙ—X, and X=¹⁸F.

12.-13. (canceled)

14. A pharmaceutical composition comprising:
a compound having the formula [I]: 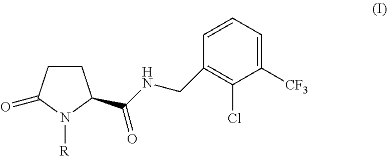
in which:
R=—(CH₂)ₙ—(O—CH₂CH₂)ₘ—Oₗ—(CH₂)ₚ—X,
l=0-1,
m=0-6,
n=2-3,
p=0-2, and
X=Br, Cl, F or ¹⁸F,
or a pharmaceutically acceptable salt or solvate thereof; and
a pharmaceutically-acceptable carrier.

15. The pharmaceutical composition of claim 14 in which the composition is in a form suitable for animal administration.

16. The pharmaceutical composition of claim 14 in which R=—(CH₂)ₙ—X, n=2-3, and X=Br, Cl or F.

17. The pharmaceutical composition of claim 16 in which n=2.

18. The pharmaceutical composition of claim 17 in which X=F.

19. The pharmaceutical composition of claim 14 in which R=—(CH₂)ₙ—X, n=2-3, and X=¹⁸F.

20.-21. (canceled)

22. A method of treating a medical condition associated with the P2X7 receptor comprising treating tissue containing the P2X7 receptor with a composition comprising:
a pharmaceutically acceptable carrier; and
a ligand compound having the formula [I]: 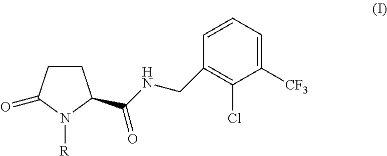
in which:
R=—(CH₂)ₙ—(O—CH₂CH₂)ₘ—Oₗ—(CH₂)ₚ—X,
l=0-1,
m=0-6,
n=2-3,
p=0-2, and
X=Br, Cl or F.

23. The method of claim 22 in which R=—(CH₂)ₙ—X and n=2-3.

24.-25. (canceled)

26. A method of imaging tissue inflammation using a radiotracer for binding to the P2X7 receptor, the method comprising:
contacting the tissue with a radiotracer composition comprising a pharmaceutically-acceptable carrier and a radiotracer compound having the formula [1]: 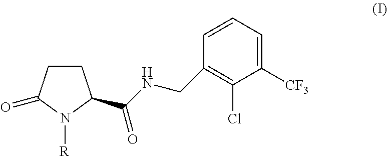
in which:
R=—(CH₂)ₙ—(O—CH₂CH₂)ₘ—Oₗ—(CH₂)ₚ—X,
l=0-1,
m=0-6,
n=2-3,
p=0-2, and
X=¹⁸F.

27. The method of claim 26 in which R=—(CH₂)ₙ-¹⁸F and n=2.

28. The method of claim 26 in which R=—(CH₂)ₙ-¹⁸F and n=3.

29. The method of claim 26 which includes:
immersing the tissue in the radiotracer composition to cause at least a portion of the radiotracer compound to bind with at least a portion of the P2X7 receptors contained by the tissue;
rinsing the tissue to remove non-bound radiotracer compound; and
imaging the rinsed tissue.
